(19) United States
(12) Patent Application Publication
(10) Pub. No.: US 2023/0078161 A1
(43) Pub. Date: Mar. 16, 2023

(54) MACHINE LEARNING-SUPPORTED PIPELINE FOR DIMENSIONING AN INTRAOCULAR LENS

(71) Applicant: Carl Zeiss Meditec AG, Jena (DE)

(72) Inventors: Hendrik Burwinkel, Munich (DE); Holger Matz, Unterschneidheim (DE); Stefan Saur, Aalen (DE); Christoph Hauger, Aalen (DE)

(73) Assignee: Carl Zeiss Meditec AG, Jena (DE)

(21) Appl. No.: 17/759,359

(22) PCT Filed: Jan. 21, 2021
(86) PCT No.: PCT/EP2021/051300

§ 371 Date: Jul. 22, 2022

(30) Foreign Application Priority Data
Jan. 24, 2020 (DE) 10 2020 101 763.4

(51) Int. Cl. A61F 2/16

(52) U.S. Cl. CPC A61F 2/1662; G06N 3/0454; A61B 2034/108

(57) ABSTRACT

The invention relates to a computer-implemented method for a machine learning-supported processing pipeline for determining parameter values for an intraocular lens to be inserted. The method comprises providing a scan result of an eye. The scan result is an image of an anatomical structure of the eye. The method further comprises determining biometric data of the eye from the scan results of an eye and using a first, trained machine learning system for determining a final position of an intraocular lens to be inserted, ophthalmological data being used as input data for the first machine learning system. The method further comprises determining a first optical power of the intraocular lens to be inserted, which is based on a physical model in which the determined final position of the intraocular lens and the determined biometric data are used as input variables for the physical model.

### CROSS-REFERENCE TO RELATED APPLICATIONS

This application is the U.S. national stage of PCT/EP2021/051300 filed on Jan. 21, 2021, which claims priority of German Patent Application DE 10 2020 101 763.4 filed on Jan. 24, 2020. The disclosures of these prior applications are considered part of the disclosure of this application and are hereby incorporated by reference in their entireties.

### TECHNICAL FIELD

The disclosure relates to determining refractive power for an intraocular lens and, in particular, to a computer-implemented method for a machine learning-supported processing pipeline for determining parameter values for an intraocular lens to be inserted, to a corresponding system, and to a corresponding computer program product for carrying out the method.

### BACKGROUND

Replacing the biological lens of an eye with an artificial intraocular lens (IOL)—for example, in the case of an (age-related) refractive error or in the case of cataracts—has become ever more common in the field of ophthalmology in recent years. In the process, the biological lens is detached from the capsular bag by way of a minimally invasive intervention and removed. The lens, which has become opacified in the case of a cataract, is then replaced by an artificial lens implant. In the process, this artificial lens implant or intraocular lens is inserted into the then empty capsular bag. Knowledge of the correct position of the intraocular lens and the necessary refractive power depend on one another.

Currently utilized IOL calculation formulas have several problems. Firstly, the position of the intraocular lens is calculated in many formulas as an effective lens position (ELP). Since this variable is not a real anatomical variable, it cannot be directly considered in a physical model to be used to calculate the complex ophthalmic optics of a patient. The ELP is calculated and optimized for the respective formula, and so there is no direct comparability between the ELPs of different formulas, and the model uses no anatomically correct optical system.

A second aspect consists of the fact that current IOL formulas use models within the prediction, the models attempting to carry out fine tuning to an availability of data by way of a few parameters. Since these are manually predefined by the developers, this is not necessarily the best representation in each case. New formulas such as the Hill RBF formula circumvent this restriction by using machine learning approaches, which are able to independently carry out optimization on the basis of the availability of data. In this case, the prediction however is based on a mass of data only, that is to say the system does not use any physical concepts and is therefore restricted in terms of its effectiveness.

In general, the current approaches exhibit no optimal combination of all available information and models present.

Proceeding from the disadvantages of the known methods for approximately determining a correct refractive power for an IOL to be inserted, an underlying object of the concept presented herein is that of specifying a method and a system for improved, integrated and fast IOL refractive power predictions for an intraocular lens, which are elegantly extendable.

### SUMMARY

This object is achieved by means of the method proposed here, the corresponding system and the associated computer program product in accordance with the independent claims. Further embodiments are described by the respective dependent claims.

According to an aspect of the present disclosure, a computer-implemented method for a machine learning-supported processing pipeline for determining parameter values for an intraocular lens to be inserted is presented. The method may include a provision of a scan result of an eye. The scan result may represent an image of an anatomical structure of the eye.

The method may furthermore include a determination of biometric data of the eye from the scan results of an eye and a use of a first trained machine learning system for determining a final position of an intraocular lens to be inserted. In this case, ophthalmological data may serve as input data for the first machine learning system. Finally, the method may include a determination of a first refractive power of the intraocular lens to be inserted, the determination being based on a physical model in which the determined final position of the intraocular lens and the determined biometric data are used as input variables for the physical model.

In accordance with another aspect of the present disclosure, a processing pipeline system for a machine learning-supported processing pipeline for determining parameter values for an intraocular lens to be inserted is presented.

The processing pipeline system may comprise a reception module configured to provide a scan result of an eye. In this case, the scan result may represent an image of an anatomical structure of the eye.

Furthermore, the processing pipeline system may comprise a determination unit configured to determine biometric data of the eye from the scan results of an eye, and a first trained machine learning system for determining a final position of an intraocular lens to be inserted. The ophthalmological data may serve as input data for the first machine learning system.

Finally, the processing pipeline system may comprise a determination unit configured to determine a first refractive power of the intraocular lens to be inserted, the determination being based on a physical model in which the determined final position of the intraocular lens and the determined biometric data are used as input variables for the physical model.

Furthermore, embodiments can relate to a computer program product able to be accessed from a computer-usable or computer-readable medium that comprises program code for use by, or in conjunction with, a computer or other instruction processing systems. In the context of this description, a computer-usable or computer-readable medium can be any device that is suitable for storing, communicating, transferring, or transporting the program code.

The computer-implemented method for determining refractive power for an intraocular lens to be inserted has a plurality of advantages and technical effects which may also apply accordingly to the associated system: the method presented here elegantly addresses the known negative properties of the disadvantages already described above. In particular, the “ZAI” algorithm, on which the method is based, facilitates an optimized calculation of the required refractive power of an intraocular lens inserted during a cataract operation. The presented algorithm allows unification of an anatomically correct prediction of the IOL position optimized by way of machine learning with a complex physical model, and allows refinement of the IOL calculation by machine learning. Hence, both an IOL position and an IOL refractive power determination can be determined in one process—or expressed differently: within a pipeline—without media disruptions.

In this case, it is possible to link both physical calculation models and machine learning concepts on the basis of clinical ophthalmological data within a pipeline for integrated determination of position and also for determining refractive power of the intraocular lens.

A machine learning system for determining refractive power for an intraocular lens to be inserted which is only based on available clinical ophthalmological data firstly would require comparatively long training times, and secondly known properties of physical models would not be able to be taken into account.

In this case, the speed advantage arising when an already trained machine learning model is retrained by better or further training data is exploited in each case. This can significantly shorten the overall training time, allowing significant economization of computational power, and hence allowing better use of the available computer capacities.

Furthermore, the use of the real physical position of the IOL allows the use of models with any desired accuracy and ultimately also the use of exact physical models. Thus, the presented method is not restricted to certain sized models and the value to be determined at the end is ultimately universal. This is in contrast to the previously used formulas as effective lens position (ELP) since this variable is not a real anatomical variable. Therefore, it also cannot be considered directly in a physical model that is used to calculate the complex ophthalmic optics of a patient.

Intraocular lenses are calculated in many formulas as effective lens position (ELP). Since this variable is not a real anatomical variable, it cannot be directly considered in a physical model to be used to calculate the complex ophthalmic optics of a patient. The ELP is calculated and optimized for the respective formula, and so there is no direct comparability between the ELPs of different formulas, and the model uses no anatomically correct optical system.

Further exemplary embodiments are presented below, which can have validity both in association with the method and in association with the corresponding system.

According to an advantageous exemplary embodiment, the method may additionally include a determination of a final refractive power of the intraocular lens by means of a second machine learning system, at least one variable from the biometric data and the first refractive power being able to be used as input variables. By way of example, the at least one variable can be the axial length of the eye. Hence, it is possible to practically carry out a transfer learning step, which uses the knowledge present in the physical model as a basis to facilitate a more accurate determination of refractive power. The second machine learning system should to this end have been trained using clinical ophthalmological data, that is to say data from earlier real patients. Such clinical ophthalmological data are typically annotated. In this way no information is lost in the pipeline: both the theoretical data of the physical model and the practical empirical data from clinical routine can be taken into account.

In this way it is also possible to include characteristic properties of certain clinics or the operating methods thereof in the pipeline. As a rule, the use of physical models does not allow this, or only allows this with the disadvantage of deviating from the known standards.

According to a further exemplary embodiment of the method, the biometric data of the eye may include at least one selected from the group consisting of the following: a pre-operational axial length, a pre-operational lens thickness, a preoperative anterior chamber depth, and an intra-operational anterior chamber depth. These may be derived from the “determining biometric data of the eye from the scan results of an eye” method step. This may be carried out in the conventional sense; however, a machine learning system can already be used to this end as well, said machine learning system determining the biometric data of the eye in a scan-direct method, in which no manual steps are required. The recorded image data of a scan result can be used directly for determining the biological parameters.

According to an advantageous exemplary embodiment of the method, a convolutional neural network, a graph attention network or a combination of the two aforementioned networks can be used in first machine learning system. By way of example, the convolutional neural network can be used to identify characteristic features in the recorded scan results and to compress the generated image data. As a result of the graph attention network it is possible to arrange known, annotated images or their compressed representation in a graph. By way of a newly recorded, current image of a patients eye, it is then possible, by way of a distance measurement to images already present in the graph, to determine the required biometric data, for example the postoperative final position of the intraocular lens. This can then be directly used in the ZAI pipeline.

According to a developed exemplary embodiment of the method, the second machine learning system can be trained in two stages, with the first training step possibly including a production—in particular by means of a computer—of first training data for a machine learning system on the basis of a first physical model for a refractive power for an intraocular lens. Subsequently, there then can be training of the second machine learning system by means of the produced first training data for the purposes of forming a corresponding learning model for determining refractive power. In this case, the hyperparameters of the machine learning system are defined by the design and the selection of the machine learning system, while the internal parameters of the machine learning system are adapted piecewise by the training.

In a second training step there then can be training of the machine learning system that was trained with the first training data using clinical ophthalmological training data for the purposes of forming a second learning model for determining refractive power. In this case, the transfer-learning principle is used; that is to say, the knowledge already learned from the physical model is now further specified by the use of real, clinical, ophthalmological training data. In this way, the training process can be accelerated significantly and fewer clinical ophthalmological training data are required since the basic structure is already preconditioned by the training using the data from the physical model.

According to an extended exemplary embodiment of the method, the one variable from the biometric data can be the pre-operational axial length. This variable can be determined elegantly using known measuring methods (e.g., by means of an OCT measurement, e.g., an A scan, a B scan, or an en-face OCT measurement).

According to an in turn extended exemplary embodiment of the method, the biometric data of the eye can be determined from the image manually or by means of a machine learning system from the provided scan results of the eye. At this point, the proposed method leaves open which partial method is used to determine the biometric data of the eye. However, a machine learning-based determination of the biometric data lends itself within the meaning of the pipeline concept.

According to an again extended exemplary embodiment of the method, further parameters of the eye can be determined when determining the final position of the intraocular lens to be inserted. These may relate to the following: The IOL position—in particular the expected final position of the IOL following a growing-in process—could be specified as a typical further parameter. Moreover, it is also possible to use a value of the IOL shift, which denotes a shift perpendicular to the optical axis. The beam path in the respectively chosen model would change depending on the shift value.

Additionally or in complementary fashion, it is also possible to use an IOL tilt value (i.e., a tilt angle of the IOL with respect to the optical axis); the beam path should be adjusted as a result of the change in this case too. The IOL type—in particular the haptic, the shape, etc. used—would also be conceivable. It may determine the position of the lens by way of the haptic/shape and thus influence the final quality of the operation (insertion of the correct IOL).

Additionally, forces on the IOL by the capsular bag, etc., should also be specified as additional parameters. This allows possible changes in the position expected in the long-term to be taken into account.

It should be pointed out that exemplary embodiments of the disclosure may be described with reference to different implementation categories. In particular, some exemplary embodiments are described with reference to a method, whereas other exemplary embodiments may be described in the context of corresponding devices. Regardless of this, it is possible for a person skilled in the art to identify and to combine possible combinations of the features of the method and also possible combinations of features with the corresponding system from the description above and below—if not specified otherwise—even if these belong to different claim categories.

Aspects already described above and additional aspects of the present disclosure become apparent inter alia from the exemplary embodiments that are described and from the additional further specific embodiments described with reference to the figures.

### BRIEF DESCRIPTION OF THE DRAWINGS

Preferred exemplary embodiments of the present disclosure are described by way of example and with reference to the following figures:

FIG. 1 illustrates a flowchart-like representation of an exemplary embodiment of the computer-implemented method for a machine learning-supported processing pipeline for determining parameter values for an intraocular lens to be inserted.

FIG. 2 depicts a cross section of a part of an eye.

FIG. 3 depicts an eye together with different biometric parameters of the eye.

FIG. 4 represents a schematic structure of essential functional blocks of the machine learning-supported pipeline for dimensioning an intraocular lens by means of the specified method.

FIG. 5 illustrates a diagram of the processing pipeline system according to the disclosure for a machine learning-supported processing pipeline for determining parameter values for an intraocular lens to be inserted.

FIG. 6 illustrates a diagram of a computer system which may additionally comprise the processing pipeline system according to FIG. 5 in full or in part.

### DETAILED DESCRIPTION OF PREFERRED EMBODIMENTS

In the context of this description, conventions, terms and/or expressions should be understood as follows:

The term “machine learning-supported processing pipeline” describes the overall concept of the method provided here and also of the system presented here. Proceeding from a recorded digital image, it is possible to determine the final refractive power of an intraocular lens to be inserted, without media disruption and without interposed manual determination of parameters. In this case, the final postoperative IOL position is used as intermediate result even without a necessary manual determination of parameters. At different points, the processing pipeline uses machine learning systems trained using real patient data. Additionally, physical models can be used. In this way, both the know-how of theoretical models and also real empirical values are included in the final determination of refractive power.

The term “intraocular lens” describes an artificial lens which can be inserted into the eye of a patient by surgery to replace the natural, biological lens.

The term “machine learning system” describes a system that is also typically assigned to a method, said system learning from examples. To this end, annotated training data (i.e., training data also containing metadata) is fed to the machine learning system in order to predict output values—output classes in the case of a classification system—that were already set in advance. If the output classes are correctly output with sufficient precision—i.e., an error rate determined in advance—the machine learning system is referred to as trained. Different machine learning systems are known. These include neural networks, convolutional neural networks (CNN) or else recurrent neural networks (RNN).

In principle, the term “machine learning” is a basic term or a basic function from the field of artificial intelligence, wherein statistical methods, for example, are used to give computer systems the ability to “learn”. By way of example, certain behavioral patterns within a specific task range are optimized in this case. The methods that are used give trained machine learning systems the ability to analyze data without requiring explicit procedural programming for this purpose. Typically, an NN (neural network) or CNN (convolutional neural network), for example, are examples of systems for machine learning, for forming a network of nodes which act as artificial neurons, and artificial connections between the artificial neurons (so-called links), wherein parameters (e.g., weighting parameters for the link) can be assigned to the artificial links. When training the neural network, the weighting parameter values adjust automatically to the links on the basis of input signals so as to generate a desired result. In the case of supervised learning, the images supplied as input values (training data)—generally (input) data—are supplemented with desired output data (annotations) in order to generate a desired output value (desired class). Considered very generally, mapping of input data onto output data is learned.

The term “neural network” describes a network made of electronically realized nodes with one or more inputs and one or more outputs for carrying out calculation operations. Here, selected nodes are interconnected by means of connections—so-called links or edges. The connections can have certain attributes, for example weighting parameter values, by means of which output values of preceding nodes can be influenced.

Neural networks are typically constructed in a plurality of layers. At least an input layer, a hidden layer, and an output layer are present. In a simple example, image data can be supplied to the input layer and the output layer can have classification results in respect of the image data. However, typical neural networks have a large number of hidden layers. The way in which the nodes are connected by links depends on the type of the respective neural network. In the present example, the prediction value of the neural learning system can be the sought-after refractive power of the intraocular lens.

The term “convolutional neural network” (CNN)—as one example of a classifier/classifier system—describes a class of artificial neural networks that are based on feedforward techniques. They are often used for image analyses using images, or the pixels thereof, as input data. The main components of convolutional neural networks are in this case convolution layers (hence the name) that allow efficient evaluation through parameter sharing. In contrast to the CNN, each pixel of the recorded image would typically be associated with an artificial neuron of the neural network as an input value in a conventional neural network.

The term “the graph attention network” (GAT) describes a neural network operating on graph-structured data. It exhibits a better behavior than the older “graphical convolutional networks” (GCNs). In the process, use is made of masked self-referenced layers of nodes which improve the known approximations in GCNs without building on computationally intensive matrix operations. A “GCN” (graphical convolutional network) would be conceivable instead of a GAT, the GCN being a certain architecture of neural networks which can also operate directly on graphs and can use the structural information present there. Alternatively, the “GraphSage” framework would also be utilizable. It is well suited to inductive representation learning in the context of large graphs. In this case, GraphSage can be used to generate low-dimensional vector representations for nodes and it is particularly useful for diagrams with comprehensive node attribute information.

Within the context of this text, the term “transfer learning” (or else curriculum learning) describes that a once developed learning model—developed by way of training the machine learning system with the training data of the physical model—is trained again. Although it is trained using related data in this second time, these related data originate from a different source than in the case of the first training. These may consist either of clinical ophthalmological data or of a second physical model, which is known for a greater accuracy of the results obtained. As a result, a second learning model is produced, which unifies in itself both the physical model parameters and the real clinical data. The “knowledge” of the respective first learning model is therefore used as a basis or starting point for the training to produce the second learning model. The learning effect of the first training can thus be transferred to the learning effect of the second training. A substantial advantage consists in the fact that the second training can be carried out comparatively more effectively, as a result of which computer resources can be economized and as a result of which the second training runs in a quicker and more targeted fashion.

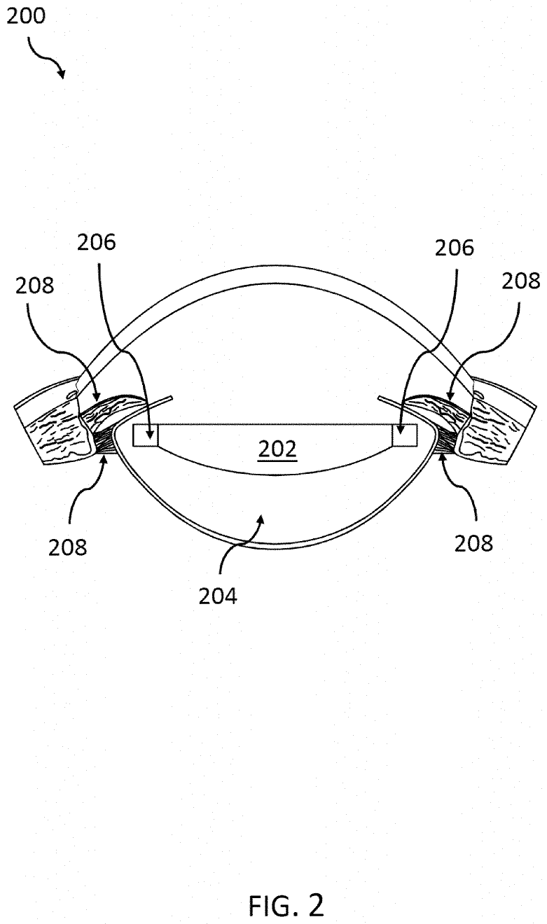

The term “parameter value” describes geometric or biometric values, or ophthalmological data of an eye of a patient. Examples of parameter values of an eye are discussed in more detail on the basis of FIG. 2.

The term “scan result” describes digital data, for example on the basis of digital images/recordings, which represent the result of an OCT (optical coherence tomography) examination on an eye of a patient.

The term “optical coherence tomography” (abbreviated OCT) describes a known imaging method of ophthalmology, for obtaining two- and three-dimensional recordings (2-D or 3-D) of scattering materials (e.g., biological tissue) with micrometer resolution. In the process, use is essentially made of a light source, a beam splitter and a sensor—for example in the form of a digital image sensor. In ophthalmology, OCT is used to detect spatial differences in the reflection behavior of individual retinal layers, and morphological structures can be represented with a high resolution.

The term “A-scan” (also referred to as axial depth scan) describes a one-dimensional result of a scan of a patients eye, which describes information about geometric dimensions and locations of structures within the eye.

The term “B-scan” describes a lateral overlay of a plurality of the aforementioned A-scans, to obtain a section through the eye. Volume views are also generable by combining a plurality of layers of the eye generated thus.

The term “en face OCT” in this case describes a method for producing transverse sectional images of the eye—in contrast to the longitudinal sectional images using the aforementioned A- or B-scans.

The term “image” or else “digital image”—e.g., from a scan—in this case describes an image representation of, or the result of generating an amount of data in the form of pixel data from, a physically existing article: by way of example, the retina of an eye in this case. More generally, a “digital image” can be understood to be a two-dimensional signal matrix. The individual vectors of the matrix can be adjoined to one another in order thus to generate an input vector for a layer of a CNN. The digital images can also be individual frames of video sequences. Image and digital image can be understood to be synonymous in this case.

The term “clinical ophthalmological training data” describes data about patients' eyes and intraocular lenses already inserted into these patients in the past. The clinical ophthalmological training data may include determined ophthalmological parameter values, such as also the refractive power and the position of the inserted lens. These data are used for the purposes of training the machine learning system which was already trained previously on the basis of data from a physical model. As a rule, the clinical ophthalmological training data are annotated.

The term “physical model” relates to a mathematical formula which relates various parameters of an eye to one another in order to undertake determinations of refractive power. A known formula is the Haigis formula.

The term “refractive power of an intraocular lens” describes the index of refraction of the IOL.

A detailed description of the figures is given below. It is understood in this case that all of the details and information in the figures are illustrated schematically. Initially, a block diagram of an exemplary embodiment of the computer-implemented method according to the disclosure for a machine learning-supported processing pipeline for determining parameter values for an intraocular lens to be inserted is illustrated. Further exemplary embodiments, or exemplary embodiments for the corresponding system, are described below:

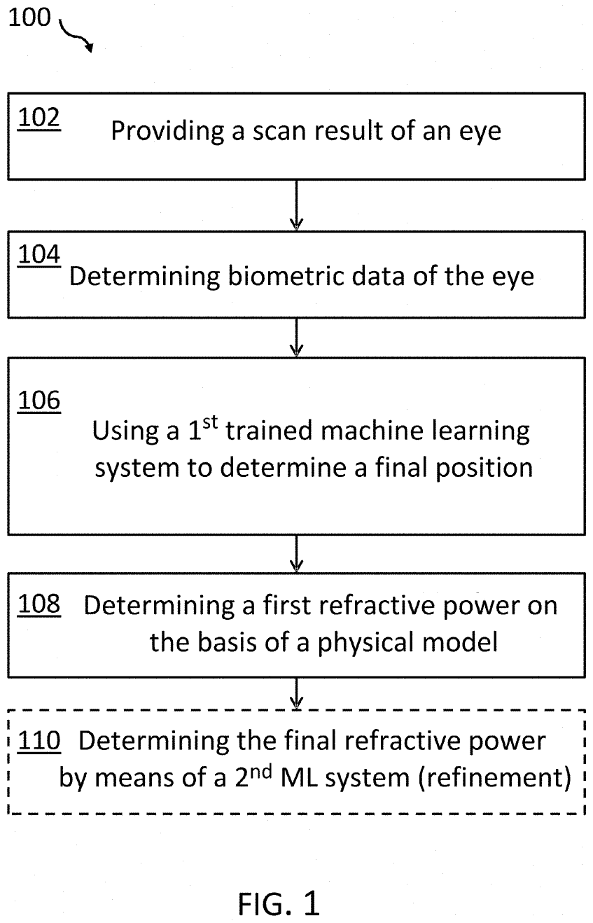

FIG. 1 illustrates a flowchart-like representation of an exemplary embodiment of the computer-implemented method 100 according to the disclosure for a machine learning-supported processing pipeline for determining parameter values for an intraocular lens to be inserted—in particular to be inserted into an eye of a patient. In this case, the method 100 includes a provision 102 of a scan result of an eye, the scan result representing an image of an anatomical structure of the eye. This can be implemented by means of OCT. An alternative—albeit less accurate—method is based on ultrasound.

The method 100 furthermore includes a determination 104 of biometric data of the eye—either conventionally or already with the aid of a machine learning system—from the scan results of an eye and a use 106 of a first trained machine learning system for determining a final position of the intraocular lens to be inserted into the eye. In this case, the long-term postoperative position of the IOL is understood to mean the final position. A determination based on a trained machine learning system may determine the long-term postoperative position directly from one (or more) recorded images of the patients eye; manual intermediate steps can be dispensed with in the process. Alternatively, the ophthalmological data—in particular those from the preceding step or those determined by “scan direct”—can serve as input data for the first trained machine learning system.

Finally, the method 100 includes a determination 108 of a first refractive power of the intraocular lens to be inserted, the determination being based on a physical model in which the determined final position of the intraocular lens and the determined biometric data are used as input variables for the physical model. In this case, the physical model is a mathematical, deterministic model.

Optionally, the determination 110 of the final refractive power can be refined or improved by means of a second machine learning system. In this case, the first refractive power and at least one variable of the biometric data—e.g., the axial length—are used as input data for the second trained machine learning system.

FIG. 2 shows a symbolic representation of a cross section of an eye 200. It is possible to identify the inserted intraocular lens 202, which has been operatively inserted into the capsular bag 204 following the removal of the natural crystalline lens. Lateral structures 206 on the intraocular lens 202 should ensure that the intraocular lens 202 is anchored truly stably in the capsular bag 204. However, a precise position of the intraocular lens 202 which sets in after a relatively long growing-in phase of several weeks, for example, could practically not be predicted to date. This is due to the fact that, inter alia, the capsular bag 204 is substantially larger than the inserted intraocular lens 202 as it previously enclosed the entire natural but now removed crystalline lens. These tendons and muscular tissue 208, which anchor the capsular bag 204 in the eye or on the skull, change after such an operation, as a result of which the size, the shape, and the position of the capsular bag 204, and hence also the position of the inserted intraocular lens 202, also change. Hence there is also a change in the distance between the inserted intraocular lens 202 and the retina situated further back in the eye. However, optimal postoperative results can only be achieved by optimal matching of the refractive power (refractive index) of the inserted intraocular lens 202 and the distance to the retina. Since the refractive power of the inserted intraocular lens 202 is normally not subsequently changeable, a prediction of the position of the inserted intraocular lens 202 is very desirable.

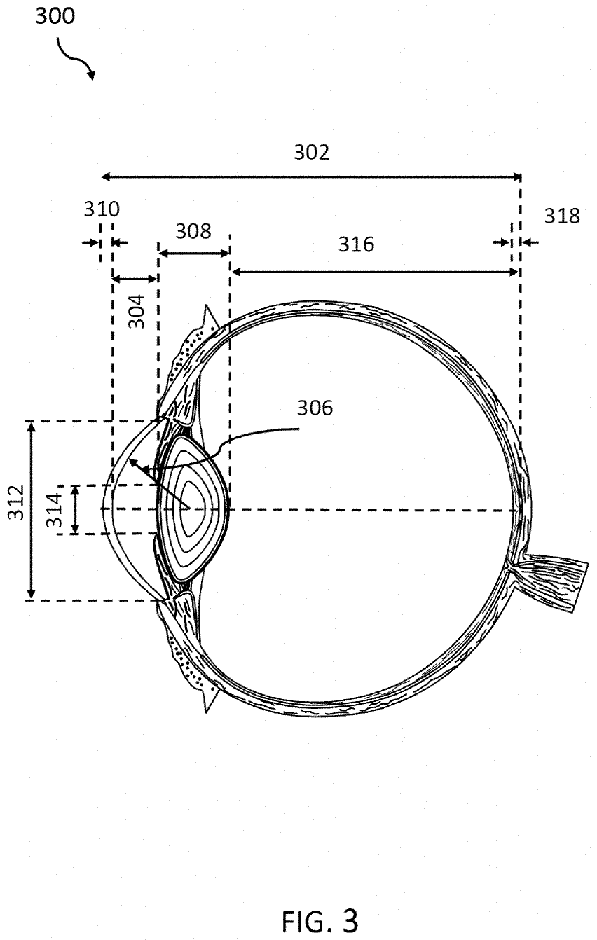

FIG. 3 depicts an eye 300 with different biometric parameters of the eye. In particular, the following parameters are represented: axial length 302 (AL), anterior chamber depth 304 (ACD), keratometry value 306 (K, radius), refractive power of the lens, lens thickness 308 (LT), central cornea thickness 310 (CCT), white-to-white distance 312 (WTW), pupil size 314 (PS), posterior chamber depth 316 (PCD), retina thickness 318 (RT). At least one of these parameters is contained both in the ophthalmological training data and in the ophthalmological data of a patient, which are each contained in the subject matter of the concept presented here.

Expressed differently, a machine learning system model incorporating known physical prior knowledge is initially created with the aid of physical models. This can be implemented by virtue of, for example, the machine learning system being pre-trained using simulation data or the training itself possibly containing physical constraints (constraint based training). Subsequently, the learning model is adapted to true anatomical variations with the aid of real clinical ophthalmological data. In this case, the chosen approach facilitates a self-learned optimization for the entire machine learning system to any availability of data (e.g., post LASIK operations). In this case, an adaptation can be carried out explicitly for each physician or for each clinic. Then, real biometric data are used as input values for the machine learning system in the application phase of said machine learning system, in order thus to determine or predict the optimized intraocular lens refractive power.

The formulation of a physical model is converted into the pure parameter form of a neural network. The latter can then independently and to the best possible extent adapt itself to a real data structure in a second training phase. Hence, any quantity of training data can be produced with the aid of the optical physical model. These contain the parameters of the eye model and the associated IOL refractive power as so-called ground truth. With the aid of the “transfer learning” concept, the model trained thus can be transferred to a more complex, physical model which produces training data according to the same concept. Hence, the neural network already has pre-trained artificial neurons and can thus adapt itself quicker and more easily to the stronger or better physical model. This curriculum learning can be carried out to a model of any strength (e.g., a ray tracing model).

In the last step the learning model then is “fine-tuned” by real biometric data of patients' eyes, with the actually used IOL refractive powers being used as ground truth. Hence, the trained model can implement in the prediction phase the prediction of the ultimately required IOL refractive power. What was found in reality is the more real data (clinical ophthalmological data) are available, the better the machine learning system can be optimized in relation to said data. Therefore, the learning model can be successively developed in accordance with the availability of data and consequently be adapted to various real data records.

In principle, the pipeline uses a machine learning model in order to use the input data from OCT measurements of the patients eye for an optimized prediction of the anatomically correct position of the intraocular lens. This position is then used in a physical model which on account of the known position of the intraocular lens can be any realistic model (e.g., a normal mathematical physical model or else ray tracing). The physical model calculates the required IOL refractive power for the eye and the result is subsequently additionally refined with the aid of machine learning in order to correct relatively small model errors in the physical model. To optimize information use, both the IOL refractive power ground truth data and the IOL position ground truth data are used for the training.

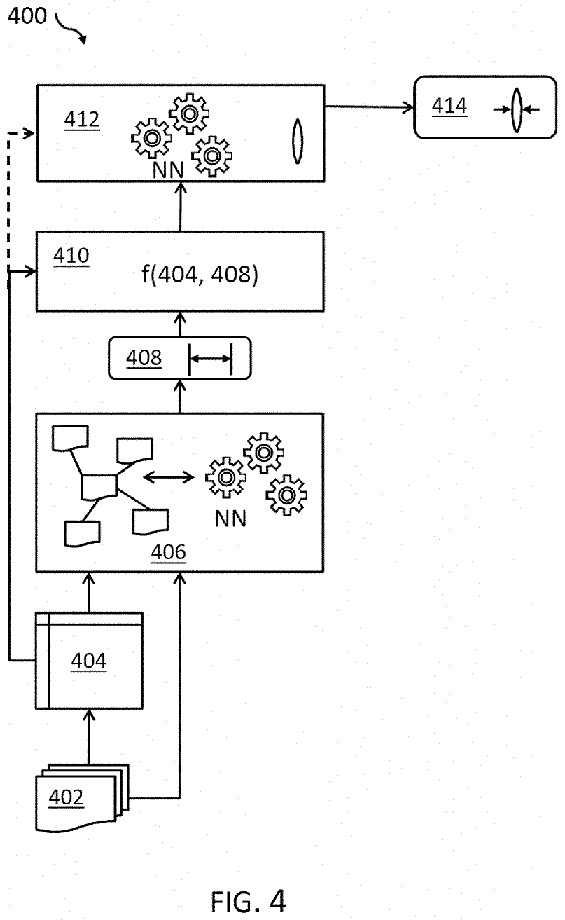

In this respect, FIG. 4 shows a schematic structure of essential functional blocks 400 of the machine learning-supported pipeline for dimensioning an intraocular lens, by means of the aforementioned method including the scan results/the images 402 of the scan of the eye. These results—in particular in the form of at least one digital image—can be used for conventional extraction of biometric data 404. At least some of these biometric data, and the scan results themselves, are supplied to a graph-based neural network 406 as input data in order to directly determine a postoperative final IOL position 408 therefrom.

Next, there is a manual, formula-based determination of refractive power 410 on the basis of a mathematical physical model. Both the extracted biometric data (or a part thereof) and the final IOL position 408 serve as the input values for this determination of refractive power 410. Additionally, a further machine learning system 412 can be used for an optimized determination of refractive power, which uses both the initially determined refractive power of the intraocular lens (as a result of the determination of refractive power 410) and the previously determined biometric data 404 (or a part thereof) as input data. The trained machine learning system 412 then supplies the ultimate final refractive power 414 on the basis of an appropriate machine learning model.

The term pipeline is disclosed directly and clearly by way of the representation of the individual steps or functional units specified in FIG. 4 since both the final postoperative IOL position 408 and the final optimized IOL refractive power can be determined by means of an integrated process with meshed partial processes.

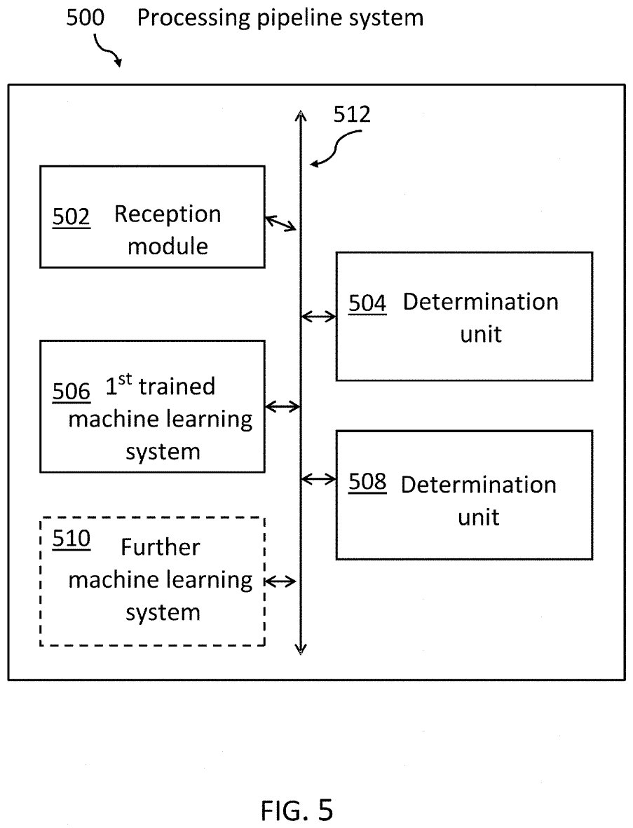

FIG. 5 illustrates—for the sake of completeness—a preferred exemplary embodiment of components of the processing pipeline system 500 for a machine learning-supported processing pipeline for determining parameter values for an intraocular lens to be inserted. The processing pipeline system 500 comprises a reception module 502 configured to provide a scan result of an eye, the scan result representing at least one image of an anatomical structure of the eye.

Furthermore, the pipeline processing system 500 comprises a determination unit 504 configured to determine biometric data of the eye from the scan results of an eye, and a first trained machine learning system 506 (cf., also, the graph-based neural network 406, FIG. 4) for determining a final position of an intraocular lens to be inserted, ophthalmological data serving as input data for the first machine learning system.

Moreover, the processing pipeline system 500 comprises a determination unit 508 (cf., also, functional block 410, FIG. 4) configured to determine a first refractive power of the intraocular lens to be inserted, the determination being based on a physical model in which the determined final position of the intraocular lens and the determined biometric data are used as input variables for the physical model.

Additionally, a further machine learning system 510 can also be used for an improved prediction of the IOL refractive power (cf., functional block 412, FIG. 4).

Express reference is made to the fact that the modules and units—in particular the reception module 502, the determination unit 504, the first trained machine learning system 506, and the determination unit 508 for determining a first refractive power—may be connected by way of electrical signal lines or via a system-internal bus system 512 in order to transfer appropriate signals and/or data from one module (one unit) to another. Furthermore, additional modules or functional units may optionally be connected to the system-internal bus system 512.

If a classification system is used as machine learning system, the predicted refractive power arises in accordance with the predicted class which is predicted with the greatest probability. Alternatively, the final refractive power of the IOL can also be implemented by means of a regression system as machine learning system with numerical output variables.

Furthermore, the system 500 may comprise an output unit (not depicted here), which is suitable for outputting or displaying the predicted final IOL refractive power, and optionally also for displaying the predicted IOL position.

FIG. 6 illustrates a block diagram of a computer system that may have at least parts of the system for determining the refractive power. Embodiments of the concept proposed here may in principle be used together with virtually any type of computer, regardless of the platform used therein to store and/or execute program codes. FIG. 6 illustrates by way of example a computer system 600 that is suitable for executing program code according to the method proposed here and may also contain the prediction system in full or in part.

The computer system 600 has a plurality of general-purpose functions. The computer system may in this case be a tablet computer, a laptop/notebook computer, another portable or mobile electronic device, a microprocessor system, a microprocessor-based system, a smartphone, a computer system with specially configured special functions or else a constituent part of a microscope system. The computer system 600 may be configured so as to execute computer system-executable instructions—such as for example program modules—that may be executed in order to implement functions of the concepts proposed here. For this purpose, the program modules may comprise routines, programs, objects, components, logic, data structures etc. in order to implement particular tasks or particular abstract data types.

The components of the computer system may comprise the following: one or more processors or processing units 602, a storage system 604 and a bus system 606 that connects various system components, including the storage system 604, to the processor 602. The computer system 600 typically has a plurality of volatile or non-volatile storage media accessible by the computer system 600. The storage system 604 may store the data and/or instructions (commands) of the storage media in volatile form—such as for example in a RAM (random access memory) 608—in order to be executed by the processor 602. These data and instructions realize one or more functions and/or steps of the concept presented here. Further components of the storage system 604 may be a permanent memory (ROM) 610 and a long-term memory 612 in which the program modules and data (reference sign 616) and also workflows may be stored.

The computer system comprises a number of dedicated devices (keyboard 618, mouse/pointing device (not illustrated), visual display unit 620, etc.) for communication purposes. These dedicated devices may also be combined in a touch-sensitive display. An I/O controller 614, provided separately, ensures a frictionless exchange of data with external devices. A network adapter 622 is available for communication via a local or global network (LAN, WAN, for example via the Internet). The network adapter may be accessed by other components of the computer system 600 via the bus system 606. It is understood in this case, although it is not illustrated, that other devices may also be connected to the computer system 600.

At least parts of the system 500 for determining refractive power of an IOL (cf., FIG. 5) may also be connected to the bus system 606.

The description of the various exemplary embodiments of the present disclosure has been given for the purpose of improved understanding, but does not serve to directly restrict the inventive concept to these exemplary embodiments. A person skilled in the art will himself/herself develop further modifications and variations. The terminology used here has been selected so as to best describe the basic principles of the exemplary embodiments and to make them easily accessible to a person skilled in the art.

The principle presented here may be embodied as a system, as a method, combinations thereof and/or else as a computer program product. The computer program product may in this case comprise one (or more) computer-readable storage medium/media having computer-readable program instructions in order to cause a processor or a control system to implement various aspects of the present disclosure.

As media, electronic, magnetic, optical, electromagnetic or infrared media or semiconductor systems are used as forwarding medium; for example SSDs (solid state devices/drives as solid state memory), RAM (random access memory) and/or ROM (read-only memory), EEPROM (electrically erasable ROM) or any combination thereof. Suitable forwarding media also include propagating electromagnetic waves, electromagnetic waves in waveguides or other transmission media (for example light pulses in optical cables) or electrical signals transmitted in wires.

The computer-readable storage medium may be an embodying device that retains or stores instructions for use by an instruction executing device. The computer-readable program instructions that are described here may also be downloaded onto a corresponding computer system, for example as a (smartphone) app from a service provider via a cable-based connection or a mobile radio network.

The computer-readable program instructions for executing operations of the disclosure described here may be machine-dependent or machine-independent instructions, microcode, firmware, status-defining data or any source code or object code that is written for example in C++, Java or the like or in conventional procedural programming languages such as for example the programming language “C” or similar programming languages. The computer-readable program instructions may be executed in full by a computer system. In some exemplary embodiments, there may also be electronic circuits, such as, for example, programmable logic circuits, field-programmable gate arrays (FPGAs) or programmable logic arrays (PLAs), which execute the computer-readable program instructions by using status information of the computer-readable program instructions in order to configure or to individualize the electronic circuits according to aspects of the present disclosure.

The disclosure presented here is furthermore illustrated with reference to flowcharts and/or block diagrams of methods, devices (systems) and computer program products according to exemplary embodiments of the disclosure. It should be pointed out that practically any block of the flowcharts and/or block diagrams can be embodied as computer-readable program instructions.

The computer-readable program instructions can be made available to a general purpose computer, a special computer or a data processing system programmable in some other way, in order to produce a machine, such that the instructions that are executed by the processor or the computer or other programmable data processing devices generate means for implementing the functions or processes illustrated in the flowchart and/or block diagrams. These computer-readable program instructions may accordingly also be stored on a computer-readable storage medium.

In this sense any block in the illustrated flowchart or block diagrams can represent a module, a segment or portions of instructions representing a plurality of executable instructions for implementing the specific logic function. In some exemplary embodiments, the functions represented in the individual blocks can be implemented in a different order—optionally also in parallel.

The illustrated structures, materials, sequences and equivalents of all means and/or steps with associated functions in the claims hereinafter are intended to apply all structures, materials or sequences as expressed by the claims.

1. A computer-implemented method for a machine learning-supported processing pipeline for determining parameter values for an intraocular lens to be inserted, the method comprising:
providing a scan result of an eye, the scan result representing an image of an anatomical structure of the eye,
determining biometric data of the eye from the scan results of an eye,
using a first, trained machine learning system for determining a final position of an intraocular lens to be inserted, ophthalmological data serving as input data for the first machine learning system,
determining a first refractive power of the intraocular lens to be inserted, the determination being based on a physical model in which the determined final position of the intraocular lens and the determined biometric data are used as input variables for the physical model, and
determining a final refractive power of the intraocular lens by means of a second, machine learning system, at least one variable from the biometric data and the first refractive power being used as input variables,
the second machine learning system being trained in two stages, with
a first training step including:
producing first training data for a machine learning system on the basis of a first physical model for a refractive power for an intraocular lens,
training the machine learning system by means of the produced first training data for the purposes of forming a first learning model for determining refractive power, and
with a second training step including:
training the machine learning system that was trained with the first training data using clinical ophthalmological training data for the purposes of forming a second learning model for determining refractive power.

2. The method of claim 1, wherein the biometric data of the eye include at least one selected from the group consisting of a pre-operational axial length, a pre-operational lens thickness, a preoperative anterior chamber depth, and an intra-operational anterior chamber depth.

3. The method of claim 1, wherein the first machine learning system is a convolutional neural network, a graph attention network or a combination of the two aforementioned networks.

4. The method of claim 1, wherein the one variable from the biometric data is the pre-operational axial length.

5. The method of claim 1, wherein biometric data of the eye are determined from the image manually or by means of a machine learning system from the provided scan results of the eye.

6. The method of claim 1, wherein further parameters of the eye are determined when determining the final position of the intraocular lens to be inserted.

7. A processing pipeline system for a machine learning-supported processing pipeline for determining parameter values for an intraocular lens to be inserted, the processing pipeline system comprising:
a reception module configured to provide a scan result of an eye, the scan result representing an image of an anatomical structure of the eye,
a determination unit configured to determine biometric data of the eye from the scan results of an eye,
a first, trained machine learning system for determining a final position of an intraocular lens to be inserted, ophthalmological data serving as input data for the first machine learning system,
a determination unit configured to determine a first refractive power of the intraocular lens to be inserted, the determination being based on a physical model in which the determined final position of the intraocular lens and the determined biometric data are used as input variables for the physical model, and
a determination unit configured to determine a final refractive power of the intraocular lens by means of a second, machine learning system, at least one variable from the biometric data and the first refractive power being used as input variables,
the second machine learning system being trained in two stages, with
a first training step including:
producing first training data for a machine learning system on the basis of a first physical model for a refractive power for an intraocular lens,
training the machine learning system by means of the produced first training data for the purposes of forming a first learning model for determining refractive power, and
with a second training step including:
training the machine learning system that was trained with the first training data using clinical ophthalmological training data for the purposes of forming a second learning model for determining refractive power.

8. A computer program product for a machine learning-supported processing pipeline for determining parameter values for an intraocular lens to be inserted, wherein the computer program product has a computer-readable storage medium having program instructions stored thereon, the program instructions being executable by one or more computers or control units and prompting the one or more computers or control units to carry out the method of claim 1.
